(19) United States
(12) Patent Application Publication
(10) Pub. No.: US 2007/0248694 A1
(43) Pub. Date: Oct. 25, 2007

(54) Anti-inflammatory properties of a standardized extract from Euphorbia hirta (PM-251) activity and for treating conditions of inflammation

(75) Inventor: Ven Subbiah, Greenville, NC (US)

Correspondence Address: DANIELS DANIELS & VERDONIK, P.A., SUITE 200 GENERATION PLAZA, 1822 N.C. HIGHWAY 54 EAST, DURHAM, NC, 27713, US

(73) Assignee: PhytoMyco Research Corporation, Greenville, NC (US)

(21) Appl. No.: 11/789,342

(22) Filed: Apr. 24, 2007

Related U.S. Application Data
(60) Provisional application No. 60/794,920, filed on Apr. 25, 2006.

(51) Int. Cl. A61K 36/47; A61K 31/7048; A61K 31/192

(52) U.S. Cl. 424/725; 514/27; 514/570

(57) ABSTRACT

An organic extract (PM-251) from a plant, Euphorbia hirta (Euphorbaceae) has been standardized. Agronomical standardization has been achieved on this plant by growing it in eastern North Carolina. Subsequently the PM 251 extract has been chemically and biologically standardized. The standardized extract has been tested for its efficacy as an anti-inflammatory product based on in vitro assays as well as in vivo (animal studies). The standardized extract has significant COX-2 (Cyclooxygenase) enzyme inhibition in vitro. PM-251 has proved to be a good anti-inflammatory agent in sub-acute rat models of inflammation in the doses of 100 mg/kbw, 200 mg/kbw and 400 mg/kbw body weights when given orally.

### CROSS REFERENCE TO RELATED APPLICATIONS

This application is related to and claims priority to U.S. Provisional Patent Application Ser. No. 60/794,920 entitled “Anti-inflammatory Properties of a Standardized Extract From Euphorbia hirta (PM-25 1),” which was filed April 25, 2006. The disclosure of that application is incorporated by reference herein in its entirety.

### BACKGROUND OF THE INVENTION

1. Field of the Invention

This invention relates to dietary supplement compositions and methods discovery, of manufacture, extraction and use thereof generally. More specifically, the invention relates in particular to compositions and methods useful for reducing the rate of inflammation, pain, and swelling in a person, to compositions and methods for affecting joint pain and swelling in a person, to compositions and methods useful to reduce the arthritis and rheumatism related conditions in a person or animal, to compositions and methods useful for reduction and control of inflammation in the treatment of persons who are in pain, and to methods of discovering compounds and compositions useful in the treatment arthritis/rheumatism related inflammation and pain and/or useful in to produce a reduction in pain in a person.

This invention also relates to dietary supplement compositions and methods generally, and in particular to compositions and methods useful for reducing inflammation as well as pain associated with any inflammation in a person. More specifically, the invention relates to the field of dietary supplement compositions derived as extracts from ethnopharmacological plants, to methods useful to identify standardized extract in such extracts that are active in the treatment of inflammation, arthritis, rheumatism and pain, and especially to compositions comprising of standardized extract (PM 251) of Euphorbia hirta plants and the compounds, Quercitrin, Ferulic acid, Gallic acid and Rhamnetin and their use in the treatment of inflammation, arthritis rheumatism and pain, such as by oral administration.

2. General Background Discussion

Rheumatoid arthritis is a chronic inflammatory disease affecting multiple tissues, but typically producing its most pronounced symptoms in the joints. It is progressive, degenerative and ultimately debilitating. The chronic inflammation in joints leads to the destruction of the soft tissue, the synovium and cartilage as well as to erosion of the articular surfaces of bones. The disease is estimated to affect over 3.2 million people in the U.S., Europe and Japan. It is more prevalent in women, who are estimated to account for a majority of the cases.

Inflammation is a natural defense of the body to protect against foreign substances or injury, but it can cause problems in certain diseases. Inflammation is a natural defense of the body to protect against foreign substances or injury, but it can cause problems in certain diseases. Inappropriate inflammation can be treated with traditional steroids, like the glucocorticoid cortisol, therapeutic proteins produced by recombinant DNA technology, and/or nonsteroidal anti-inflammatory drugs (NSAIDs). Several interesting therapeutic proteins have resulted from recombinant DNA and monoclonal antibody technology, including Embrel®, Remicade®, and recombinant protein C. Unfortunately, many of the therapeutic treatments involve additional safety risks, partly because of the manner in which they suppress the immune system. In fact, all the more powerful anti-inflammatory agents (e.g., glucocorticoids) increase the risk of infection because of such suppression.

Prostaglandins are a related family of chemicals that are produced by the cells of the body and serve many essential functions including the promotion of pain, inflammation, and fever. Additionally, some prostaglandins support the function of platelets, necessary for blood clotting, and protect the stomach lining from the damaging effects of acid. Prostaglandins are produced within the body's cells by the enzyme cyclooxygenase(COX).

Cyclooxygenase is an enzyme naturally present in our body. Scientists discovered there were two forms of this cyclooxygenase enzyme: COX-1 and COX-2. COX-1 enzymes are produced widely throughout the body and is involved in the regulation of day-to-day cellular and metabolic activities such as maintaining stomach lining integrity, regulating blood flow within the kidneys and balancing platelet function. COX-1 enzymes are present in the body always and should not be inhibited. COX-2 enzymes are necessary for inducing pain, and COX-2 inhibitors became available in 1998. COX-2 also produces prostaglandin molecules, but the COX-2 enzyme is located specifically in areas of the body that are responsible for inflammation and not in the stomach. The COX-2 enzyme is present in our bodies, ideally on a limited basis; however, factors such as diet, stress, and injury can influence COX-2 production. When COX-2 is produced on a continual basis, constant pain ensues. Therefore, inhibiting COX-2 is an option for muscle pain management.

It is important to understand that Cyclooxygenase-1 (COX-1) and -2 (COX-2) enzymes are responsible for the conversion of arachidonic acid, a lipid present in the cell, to prostaglandins. Prostaglandins in turn cause inflammatory responses in the body. Inhibition of COX-1 enzyme may result in the formation of ulcers in many human and hence the selective inhibition of COX-2 enzyme by compounds has a major advantage over non-selective nonsteroidal anti-inflammatory drug (NSAIDs) sold over the counter (OTC). It is important to note that over expression of COX-2 enzyme was observed not only in inflamed cells but also by various types of tumor cells. Hence, COX-2 inhibitors with little or no COX-1 activity are of great interest for the chemoprevention of cancer.

3. Description of Background Art

It is known that the plant Euphorbia hirta L., belongs to the family Euphorbiaceae. Synonyms include E. pilulifera L. and Chamaesyce hirta (L.) Millsp. It is commonly known as Snake weed. Whole aerial parts of the plant has been used in ethnic preparations. The plant is an erect rigid herbs; branches ascending; stem terete below, angular above, hispid, with long, often yellowish hairs. Leaves are 1.5-2×0.5-0.7 cm, opposite, elliptic-oblong or oblong-lanceolate, serrate-crenate, apex obtuse or acute, base rounded, unequal hairy; hairs long, hispid beneath, floral leaves small, opposite. Cyathia is crowded, in axillary, small cymes. It is involucre with 4 stalked glands. Capsule is 0.5 mm long, 3-valved, adpressed-pubescent; the number of seeds is 3. Flowering and fruiting will occur throughout the season. The plant is native to Asia and distributed all over tropical and subtropical regions. The plant has been successfully grown in eastern North Carolina. Agronomic conditions, such as sources of germplasm and cultivation, harvest and processing have been completed in eastern North Carolina.

E. hirta has, in the past, been used mainly in treating respiratory ailments (e.g., asthma, bronchitis, coughs, and hay fever); and also tumors. The leaves are edible. The latex of the plant is used as an application for warts and in Africa the latex of the plant is used to treat bee and scorpion sting bites/

### BRIEF SUMMARY OF THE INVENTION

The present invention relates to the use of Euphorbia hirta standardized extract as selective cyclooxygenase-2 (COX-2) inhibitors. The Euphorbia hirta standardized extract also showed significant activity in inhibiting inducible nitric oxide synthase (iNOS) in the Griess assay. This enzyme produces nitric oxide (NO), a molecule involved in anti-oxidant pathways that is responsible for many of the pathologies associated with the inflammatory responses.

The present invention relates to a method for selectively inhibiting COX-2 enzyme relative to COX-1 enzyme which comprises providing an effective amount of a standardized Euphorbia hirta extract so as to produce the COX-2 inhibition. Preferably the Quercitrin, Ferulic acid, Gallic acid and Rhamnetin are present in plant material. In particular, the present invention relates to a method for selectively inhibiting COX-2 enzyme relative to COX-1 enzyme which comprises providing an effective amount of an standardized extract of E. hirta so as to produce the COX-2 enzyme inhibition. The inhibition can be in vitro; however, preferably inhibition is in vivo in a mammal. Cyclooxygenase-2 (COX-2) is a bifunctional enzyme that exhibits both cyclooxygenase and peroxidase activities. To evaluate inhibition of COX-2 by PM 251, the peroxidase activity was assayed colorometerically by monitoring the appearance of oxidized tetramethyl-p-phenyldiamine. DUP-697, a known COX-2 inhibitor, was used as an internal control and as expected inhibited COX-2 with an 150 of about 200 nM.

### BRIEF DESCRIPTION OF THE DRAWING

FIG. 1 is a graph showing fingerprinting of standardized extract from Euphoria hirta (PM 251-3); and

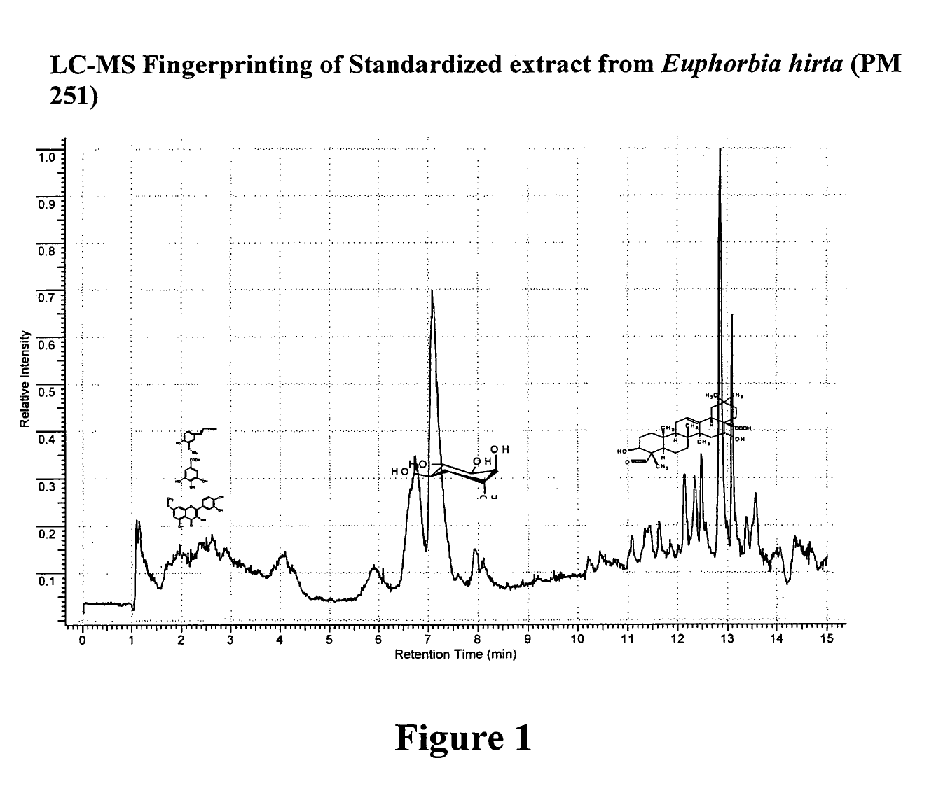

FIG. 2 is a graph showing COX-2 enzyme activity by PM 251 in vitro.

### DETAILED DISCUSSION OF THE INVENTION

Having briefly generally described the invention, the following is a more detailed discussion.

In one aspect, the invention relates to a process for the identification of a composition or compound useful in the treatment of an inflammation, inflammation related arthritis, rheumatism, and pain in humans. The process involves preparing an assay by obtaining an extract of an ethnobotanical plant. The activity of the extract is evaluated and an assay selected from a group made up of a COX-2 enzyme inhibition, COX-1 enzyme inhibition assay, and an assay that measures the amount of inhibition of Cyclooxygenase enzymes, or combinations thereof. Preferably, the inhibitor is a standardized plant preparation of Euphorbia hirta and other Euphorbia species containing Quercitrin, Ferulic acid, Gallic acid and Rhamnetin analogs. The inhibitor is preferably selected from the group made up of Quercitin, Ferulic acid. Gallic acid and Rhamnetin.

In accordance with the invention, it has been discovered that compositions generally represented by the compounds described previously are useful for treating conditions not previously known to be susceptible as an inflammation with complications discussed herein. In one aspect, a method of preventing or treating an inflammatory disease is described. Such a method is applied to a mammal in need of such treatment and includes the steps of administrating a COX-2 inhibitory effective amount of a composition of the previously described formula. For therapy, administration of one or more compounds present in the standardized extract is useful. An oral tablet or capsule of the standardized extract with 1-2% of Quercitin, 0.5-1.5% Ferulic, 0.1-0.5% Gallic acid and 0.3-1.0% Rhamnetin or a similar formulation with suitable excipients, is most effective, although an higher percentage of all four compounds (Quercitin, Ferulic acid. Gallic acid and Rhamnetin) also useful. These amounts are by weight.

Typically, the parenteral dose will be about 0.01 to about 50 mg/kg; preferably between 0.1 and 20 mg/kg, in a manner to maintain the concentration of drug in the plasma at a concentration effective to inhibit the COX-2 enzyme. The standardized extract is administered one to three times daily at a level to achieve a total daily dose of about 20 to about 80 mg/kg/day. The precise amount of the standardized extract used in the present method which is therapeutically effective, and the route by which such compound is best administered, is readily determined by one of ordinary skill in the art by comparing the blood level of the agent to the concentration required to have a therapeutic effect.

Thus, it is clear from the above-discussion that compounds as identified herein possess COX-2 inhibitory activity and are useful for the treatment of the disease other than possible with just antagonistic activity previously known. In accordance with the invention, mammals are treated with compositions which are used as inhibitors of COX-2 with anti-inflammatory, anti-proliferative as well as by hyperplasia, all of which are often observed in rheumatoid arthritis and other inflammatory diseases.

In accordance with the method of selectively inhibiting COX-2 enzymes relative to COX-1 enzymes the invention involves providing an effective amount of a PM-251 standardized extract to cause COX-2 inhibition. More specifically, Quercitrin, Ferulic acid, Gallic acid and Rhamnetin are present in plant material of Euphorbia hirta and extracted therefrom. In a preferred aspect, the method involves providing an effective amount of an isolated and purified Quercitrin, Ferulic acid, Gallic acid and Rhamnetin compounds or mixtures thereof, extracted from the Euphorbia hirta to produce the COX-2 enzyme inhibition.

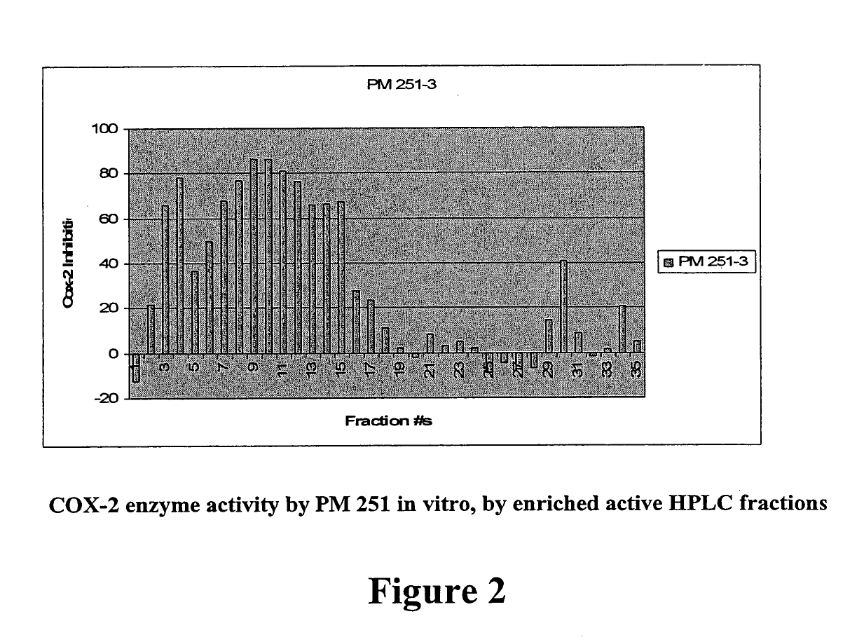

In a yet more preferred aspect, the inhibition is caused in vitro (see FIG. 2). In yet another alternative and preferred aspect, the inhibition is caused in vivo in a mammal. The following describes such applications in vitro and in vivo.

Dose Levels
Control: One ml of 1% gum acacia
Standard: 100 mg/kbw (kilogram body weight) in acute models and sub-acute models.
T1:100 mg/kbw and T2:200 mg/kbw in Carrageenin and Turpentine models and T1:

200 mg/kbw and T2:400 mg/kbw in Cotton pellet granuloma and Formalin induced arthritis model.

Dosing

| | |
|---|---|
| Route: | Oral with the help of mouth gag and ryles tube |
| Frequency: | Only one dose of drug for acute models and once daily for |
| | 10 days for the sub-acute model. |
| Procedure: | The control, the standard and test articles (T1 and T2) were |
| | administered to the rats orally via a mouth gag and ryles |
| | tube. |

Justification for Route, Dose Levels and Dosing Schedule
The oral route is common and reliable in humans.
The frequency of dosing is the convention for general potency and toxicity studies and conventional dose regime was followed, 1 ml of 1% gum acacia in control group and 100 mg/kbw in acute models and sub-acute model of the standard group and T1:100 mg/kbw and T2:200 mg/kbw in Carrageenin and Turpentine models and T1:200 mg/kbw and T2:400 mg/kbw in Cotton pellet granuloma and Formalin induced arthritis model. All drugs (dose) were given only once a day.

2. Method of performance study
Main assays: The test article, the standard and control were administered: a. Carrageenin induced rat paw edema: The respective drug was administered in single dose through mouth 1 hour before the administration of phlogistic agent. The method used comprises of the study of the acute inflammatory reaction induced by injecting the phlogistic agent carrageenin into the subplantar surface of the right hindpaw of the rat. The volume of the right hindpaw was measured at ‘0’ hour and at the end of ‘4’ hours after the administration of phlogistic agent with the help of Mercury plethysomograph and the difference between ‘0’ hour and ‘4’ hours reading gives the actual edema. Lesser the mean paw volume more was the anti-inflammatory potential and vice versa.
b. Turpentine induced arthritis: The respective drug was administered in single dose through mouth 1 hour before the administration of phlogistic agent. This is one of the commonly employed models to induce arthritis in the rat; 0.01 ml turpentine was injected to the right knee joint. The maximum diameter of the right knee joint was measured at ‘0’ hour and at the end of ‘4’ hours after the injection with the help of screw gauge and the difference between ‘0’ hour and ‘4’ hour reading gave the actual arthritis. Lesser the mean diameter right knee joint more the anti-inflammatory potential and vice versa.
c. Formalin induced experimental peritonitis: The respective drug was administered in single dose through mouth 1 hour before the administration of phlogistic agent by injecting 1 ml of 1% formalin in to the peritoneal cavity. This was chosen for inducing experimental inflammation and the parameter for assessment of anti-inflammatory activity was by measuring the volume of the exudate formed after 4 hours by using a measuring jar after opening the abdominal cavity. Lesser the mean exudates more is the anti-inflammatory potential and vice versa.
d. Cotton wool pellet induced granuloma: The respective drug was administered by mouth in single dose one hour prior to the cotton pellet implantation on the first day and subsequently every day for next five days. The extent of formation of foreign body granuloma (granulation tissue) is the main criterion for evaluating anti-inflammatory activity. Sterile cotton pellets (10 mg) were introduced into the sub-cutaneous plain of each axilla and groin and then sutured. The experiment was continued for five days and on the sixth day the rats were sacrificed and the sutures were opened, the cotton pellets were removed and dried in hot air oven at 60° C. for six hours and weighed. The difference in the weight of the cotton pellet prior to implantation into sub-cutaneous plain (10 mg) and after drying indicates the total granulation tissue formed. Lesser the granulation tissue formed more is the anti-inflammatory potential.
   The known volume (predetermined based on the rat weight) of the appropriate test material was delivered into the stomach of each rat via a mouth gag and ryles tube. The flow path was gently rubbed while administering the test material. Data of all the above mentioned models are presented in Table Ia, Ib, Ic, Id in tabular form respectively.
   The above mentioned data suggest that PM 251 in the dose of 100 mg/kbw (in Carrageenan induced rat paw edema and Turpentine induced arthritis) and 200 mg/kbw (Ti) (in Formalin induced experimental peritonitis and Cotton pellet induced granuloma) body weight given orally, showed significant anti-inflammatory activity when compared with Indomethacin in Turpentine induced arthritis, Formalin induced experimental peritonitis and Cotton pellet induced granuloma and showed almost equipotent activity with PM 251 in the dose of 200 mg/kbw and 400 mg/kbw (T2), both in acute and sub-acute models of experimental inflammation.
   Compound PM 251 in its (T2) dose with respect to control (Table IIb and FIG. Ib) has shown high anti-inflammatory activity in:
   1. Carrageenin induced rat paw edema model (81.47%)
   2. Formalin induced experimental peritonitis (130.56%) and very high activity
   3. Cotton pellet induced granuloma model (146.27%)
   But has shown moderate anti-inflammatory activity in:
   1. Turpentine induced arthritis (59.31%).
   The same compound with the same dose when compared with Indomethacin (standard)
   (Table IId and FIG. Id), anti-inflammatory activity was very less in:
   1. Carrageenin induced rat paw edema model (20.46%);
   It has showed good anti-inflammatory activity in two acute and the sub-acute model:
   1. Turpentine induced arthritis (69.82%),
   2. Formalin induced experimental peritonitis (94.44%)
   3. Cotton pellet induced granuloma model (61.57%).
   PM 251 in the dose of 200 mg/kbw and 400 mg/kbw (T2), both in acute and sub-acute models of experimental inflammation respectively is found to equipotent compared with PM 251 in the dose of 100 mg/kbw and 200 mg/kbw (T1) body weight (Table IIf and FIG. If), the acute and the sub-acute models of experimental inflammation. This concludes that with administration of test drug (both T1 and T2) produce equipotent anti-inflammatory activity.
   In conclusion with the available statistics of the study PM 251 has proved to be a good anti-inflammatory agent in acute and sub-acute models of inflammation in the doses of 100 mg/kbw, 200 mg/kbw and 400 mg/kbw body weights when given orally, except in Carrageenan induced rat paw edema model when compared with Standard.
   No dose dependent anti-inflammatory effect was observed in T1 and T2 groups when compared with Control and Standard.

Note: T1=100 mg/kbw and T2=200 mg/kbw in Carrageenin induced rat paw edema model and Turpentine induced arthritis and T1=200 mg/kbw and T2=400 mg/kbw in Formalin induced experimental peritonitis and Cotton pellet induced granuloma.

Study model: Carrageenin induced rat paw edema**

| | | | | Mean volume | % inhibition | Inhibitory |
|---|---|---|---|---|---|---|
| Sl. | | Type of drug | Dose | (in ml.) (±) | with respect to | Potency |
| No. | Group | administered | administered | SEM | control | Values |
| 1. | Control | Gum acacia | 1 ml of 1% | 3.52 ± 0.32* | N.A.*** | N.A.*** |
| 2. | Standard | Indomethacin | 100 mg/kbw | 0.88 ± 0.52* | 398.11 | 3.98 |
| 3. | T1 | PM 251 | 100 mg/kbw | 4.42 ± 0.68* | 79.62 | 0.79 |
| 4. | T2 | PM 251 | 200 mg/kbw | 4.32 ± 0.84* | 81.47 | 0.81 |

*P values: <0.05
**Animals used Wister strain - Albino rats weighing 120 to 170 gms
***N.A: Not applicable

TABLE Ib
Study model: Turpentine induced arthritis**

| | | | | Mean volume | | Inhibitory |
|---|---|---|---|---|---|---|
| Sl. | | Type of drug | Dose | (in mm.) (±) | % inhibition with | Potency |
| No. | Group | administered | administered | SEM | respect to control | Values |
| 1. | Control | Gum acacia | 1 ml of 1% | 1.32 ± 0.19* | N.A.*** | N.A.*** |
| 2. | Standard | Indomethacin | 100 mg/kbw | 1.55 ± 0.19* | 84.95 | 0.85 |
| 3. | T1 | PM 251 | 100 mg/kbw | 1.84 ± 0.17* | 71.75 | 0.72 |
| 4. | T2 | PM 251 | 200 mg/kbw | 2.22 ± 0.23* | 59.31 | 0.59 |

*P Values: <0.05
**Animals used Wister strain - Albino rats weighing 120 gms to 180 gms
***N.A: Not applicable

TABLE Ic
Study model: Formalin induced peritonitis**

| | | | | Mean volume | | Inhibitory |
|---|---|---|---|---|---|---|
| Sl. | | Type of drug | Dose | (in ml.) (±) | % inhibition with | Potency |
| No. | Group | administered | administered | SEM | respect to control | Values |
| 1. | Control | Gum acacia | 1 ml of 1% | 0.78 ± 0.18* | N.A.*** | N.A.*** |
| 2. | Standard | Indomethacin | 100 mg/kbw | 0.57 ± 0.14* | 138.24 | 1.38 |
| 3. | T1 | PM 251 | 200 mg/kbw | 0.62 ± 0.25* | 127.03 | 1.27 |
| 4. | T2 | PM 251 | 400 mg/kbw | 0.60 ± 0.17* | 130.56 | 1.30 |

*P Values: <0.05
**Animals used Wister strain - Albino rats weighing 120 gms to 180 gms
***N.A: Not applicable

TABLE 1d
Study model: Cotton pellet induced granuloma**

| | | | | | % inhibition | Inhibitory |
|---|---|---|---|---|---|---|
| Sl. | | Type of drug | Dose | Mean volume | with respect to | Potency |
| No. | Group | administered | administered | (in mg.) (±) | control | Values |
| 1. | Control | Gum acacia | 1 ml of 1% | 15.54 ± 0.52* | N.A.*** | N.A.*** |
| 2. | Standard | Indomethacin | 100 mg/kbw | 6.54 ± 0.36* | 237.58 | 2.38 |
| 3. | T1 | PM 251 | 200 mg/kbw | 11.63 ± 0.93* | 133.69 | 1.34 |
| 4. | T2 | PM 251 | 400 mg/kbw | 10.63 ± 1.14* | 146.27 | 1.46 |

*P Values: <0.001
**Animals used Wister strain - Albino rats weighing 120 gms to 200 gms
***N.A: Not applicable.

Conclusion:
PM 251-3 in the dose of 200 mg/kbw and 400 mg/kbw (T2), both in acute and sub-acute models of experimental inflammation respectively is found to equipotent compared with PM 251 in the dose of 100 mg/kbw and 200 mg/kbw (T1) body weight (Table IIf and FIG. If), the acute and the sub-acute models of experimental inflammation. This concludes that with administration of test drug (both T1 and T2) produce equipotent anti-inflammatory activity. In conclusion with the available statistics of the study PM 251 has proved to be a good anti-inflammatory agent in acute and sub-acute models of inflammation in the doses of 100 mg/kbw, 200 mg/kbw and 400 mg/kbw body weights when given orally, except in Carrageenan induced rat paw edema model when compared with Standard.
No dose dependent anti-inflammatory effect was observed in T1 and T2 groups when compared with Control and Standard.
Note: T1=100 mg/kbw and T2=200 mg/kbw in Carrageenin induced rat paw edema model and Turpentine induced arthritis and T1=200 mg/kbw and T2=400 mg/kbw in Formalin induced experimental peritonitis and Cotton pellet induced granuloma.

Yet in a more specific and alternative aspect, the invention also relates to a composition which includes Quercitrin, Ferulic acid, Gallic acid and Rhamnetin mixtures, increased over an amount occurring in nature, and placed in a pharmaceutically acceptable carrier such as, 1% gum acacia, although other like carries will suffice.

Yet still further, the composition includes isolated and purified Quercitrin, Ferulic acid, Gallic acid and Rhamnetin compounds or mixtures thereof which have been present and have been extracted from Euphorbia hirta.

The following is a detailed example wherein PM-251-3 is standardized and the active molecules identified.

### EXAMPLE

### Procedure: Standardization of PM 251 and Identification of the Active Molecules Protocol

The aerial part of Euphorbia hirta were cut into small pieces and were dried in an Drying oven at 45 C to remove all traces of moisture. The dried materials were stored at −20 C freezers before processing.

The dried samples were Pulverized using Wiley mill with appropriate filters to obtain 20-40 micron particle size. Again, the samples were stored at −20 C in air tight zip log bags.

About 50 gram of sample was systematically extracted on a Soxhlet extractor using methanol. The methanol fraction was dried using Flash rotoevaporator to remove all solvents and dried samples were freezed and freeze-dried to obtain completely dry powder. The powders were transferred to pre-weighed Scintillation vials and stored at −20 C.

The methanolic crude extract was subjected to Flash column chromatography using Chloroform-methanol gradient and the active pooled fractions (Fractions 30-47) rich with Quercitin, Ferulic acid and Gallic acid was designated as PM 251-3 standardized extract and subjected to fingerprinting on an LC-MS.

The PM 251-3 was chromatographed on a hypersil C18 reversed phase column (100×2.1 mm, 5 μm), eluted with water-acetonitrile gradient with a flow rate of 0.6 ml/min. The sample was monitored based on the UV character a swell as by mass detection.

For LC, LC pumps: Shimadzu LC-6 binary high pressure system with CTC PAL injector was used. C1 8 (reversed phase) Agilent Zorbax XDB colum with a dimension, 21.2×100 mm. The solvent system consisted of water with 0.05% TFA and Methanol with 0.05% TFA. The injection volume: 500 uL. The fractions were collected using a fraction collector (Advantec set to time based fractionation starting at 0.8 min with 0.20 min collection steps). The LC/MS/MS data was acquired for PM 251-3 using both electrospray positive and negative ion detection. The LC/MS detection included UV and total ion current chromatogram for the electrospray positive ion LC/MS/MS analysis. LC/MS mass spectra for the most intense peaks in the chromatogram were analyzed. The LC/MS/MS data acquired from the standards of Quercitin, Ferulic acid and Gallic acid using the same conditions were used to quantify Quercitin, Ferulic acid and Gallic acid in the standardized PM 251-3. PM 251 has been standardized and marker compounds assigned to the extracts based on COX-2 enzyme inhibition properties. The structures of the compounds exhibiting COX-2 inhibition have been identified based on their peak elution on the LC-MS profile (see figures).

Having thus generally described the invention, the same will be better understood from the appended claims in which it is set forth in a non-limiting manner.

What is claimed is:

1. A process for the identification of a composition or compound useful in the treatment of an inflammation, inflammation related arthritis, rheumatism and pain in humans, comprising an assay comprising:
obtaining an extract of an ethnobotanical plant, and
evaluating the activity of the extract in an assay selected from the group consisting of a COX-2 enzyme inhibition, COX-1 enzyme inhibition assay, an assay that measures the amount of inhibition of Cyclooxigenase enzymes, and combinations thereof.

2. The method of claim 1, wherein the inhibitor is a standardized plant preparation of at least one of Euphorbia hirta and other Euphorbia species containing Quercitrin, Ferulic acid, Gallic acid, Rhamnetin or analogs thereof.

3. The method of claim 1, wherein the inhibitor is selected from the group consisting of Quercitrin, Ferulic acid, Gallic acid and Rhamnetin.

4. A method of preventing or treating an inflammatory disease in a mammal in need of such treatment, comprising:
administering a COX-2 inhibitory effective amount of a composition containing Quecitrin, Ferulic acid, Gallic acid, Rhamnetin or analogs thereof.

5. The method of claim 4, for selectively inhibiting COX-2 enzyme relative to COX-1 enzyme, comprising administrating an effective amount of a PM 215 standardized extract so as to produce the COX-2 inhibition.

6. The method of claim 4, wherein the Quercitrin, Ferulic acid, Gallic acid and Rhamnetin are present and extracted from plant material of Euphorbia hirta.

7. The method of claim 4 for selectively inhibiting COX-2 enzyme relative to COX-1 enzyme, comprising administering an effective amount of an isolated and purified Quercitrin, Ferulic acid, Gallic acid and Rhamnetin compounds or mixtures thereof present in Euphorbia hirta so as to produce the COX-2 enzyme inhibition.

8. The method of claim 4, wherein the administering to cause inhibition is conducted in vitro.

9. The method of claim 7, wherein the administering to cause inhibition is conducted in vitro.

10. The method of claim 4 wherein the administering to cause inhibition is conducted in vivo in a mammal.

11. The method of claim 7 wherein the administering to cause inhibition is conducted in vivo in a mammal.

12. A composition which comprises:
Quercitrin, Ferulic acid, Gallic acid and Rhamnetin mixtures thereof increased over an amount occurring in nature; and
a pharmaceutically acceptable carrier, wherein the composition selectively inhibits COX-2 enzyme relative to COX-1 enzyme.

13. The composition of claim 11 which comprises:
an isolated and purified Quercitrin, Ferulic acid, Gallic acid and Rhamnetin compounds or a mixtures thereof present in Euphorbia hirta; and
a pharmaceutically acceptable carrier, wherein the composition selectively inhibits COX-2 enzyme relative to COX-1 enzyme.
